(19) United States
(12) Patent Application Publication
(10) Pub. No.: US 2025/0241752 A1
(43) Pub. Date: Jul. 31, 2025

(54) Valve and Delivery Systems for Transcatheter Valve-in-Valve Implantation

(71) Applicant: St. Jude Medical, Cardiology Division, Inc., St. Paul, MN (US)

(72) Inventors: Michael Shane Morrissey, St. Paul, MN (US); Tyler Govek, St. Louis Park, MN (US); Victoria Schuman, Minneapolis, MN (US); Robert Schulzetenberg, Columbia Heights, MN (US)

(73) Assignee: St. Jude Medical, Cardiology Division, Inc., St. Paul, MN (US)

(21) Appl. No.: 18/945,153

(22) Filed: Nov. 12, 2024

Related U.S. Application Data
(60) Provisional application No. 63/626,679, filed on Jan. 30, 2024.

(51) Int. Cl. A61F 2/24; A61M 25/10

(52) U.S. Cl. CPC A61F 2/2433; A61M 25/10182; A61M 25/10184; A61M 2205/3331

(57) ABSTRACT

A method of fracturing a previously-implanted prosthetic surgical heart valve may include advancing a non-compliant balloon of a balloon catheter through a patient's vasculature until the balloon is positioned at least partially within the previously-implanted prosthetic surgical heart valve. Inflation media may be advanced through an inflation lumen and into the balloon to inflate the balloon. A pressure within the inflation lumen may be measured during advancing the inflation media. A volume of inflation media advanced through the inflation lumen and into the balloon may be measured. A relationship between the measured pressure and the measured volume of inflation may be monitored, as the inflation media is advanced into the balloon, to identify distinct phases of balloon inflation.

This application claims priority to the filing date of U.S. Provisional Patent Application No. 63/626,679, filed Jan. 30, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

The present disclosure addresses problems and limitations associated with the related art.

### SUMMARY OF THE DISCLOSURE

According to one example of the disclosure, a method of fracturing a previously-implanted prosthetic surgical heart valve includes advancing a non-compliant balloon of a balloon catheter through a patient's vasculature until the balloon is positioned at least partially within the previously-implanted prosthetic surgical heart valve. Inflation media may be advanced through an inflation lumen and into the balloon to inflate the balloon. A pressure within the inflation lumen may be measured during advancing the inflation media. A volume of inflation media advanced through the inflation lumen and into the balloon may be measured. A relationship between the measured pressure and the measured volume of inflation may be monitored, as the inflation media is advanced into the balloon, to identify distinct phases of balloon inflation. Monitoring the relationship between the measured pressure and the measured volume of inflation may include viewing a real-time pressure-volume curve provided on a display based on the measured pressure and the measured volume. Identifying distinct phases of balloon inflation may include identifying a first phase of inflation corresponding to initial balloon filling phase in which the measured pressure stays substantially constant as the measured volume increases, the pressure-volume curve having a first slope during the first phase of inflation. Identifying distinct phases of balloon inflation may include identifying a second phase of inflation corresponding to additional balloon filling phase in which the measured pressure increases as the measured volume increases, the pressure-volume curve having a second slope during the second phase of inflation, the second slope being greater than the first slope. Identifying distinct phases of balloon inflation may include identifying a third phase of inflation corresponding to contact between the balloon and the previously-implanted prosthetic surgical valve in which the measured pressure increases as the measured volume increases, the pressure-volume curve having a third slope during the third phase of inflation, the third slope being greater than the second slope. Identifying distinct phases of balloon inflation includes identifying a fourth phase of inflation corresponding to a frame of the previously-implanted prosthetic surgical valve being fractured in which the measured pressure decreases as the measured volume increases. The method may include stopping advancing inflation media through the inflation lumen after identifying the fourth phase of inflation. Advancing inflation media through the inflation lumen and into the balloon to inflate the balloon may be performed with a motorized inflation system, and the motorized inflation system may automatically stop advancing inflation media through the inflation lumen after identifying the fourth phase of inflation. A critical balloon pressure threshold may be identified. The method may include stopping advancing inflation media through the inflation lumen if the measured pressure reaches the critical pressure balloon pressure threshold before the balloon fractures the previously-implanted prosthetic surgical heart valve.

According to an example of the disclosure, a balloon catheter system is configured to fracture a previously-implanted prosthetic heart valve, and the balloon catheter system includes a motorized inflation system configured to (i) operably couple to a syringe filled with inflation media, (ii) pressurize the syringe to advance inflation media out of the syringe, and (iii) measure a volume of inflation media advanced out of the syringe. A balloon catheter may include a balloon at a distal end thereof, the balloon configured to be in fluid communication with the syringe via an inflation lumen when the syringe is operably coupled to the motorized inflation system and when the balloon catheter is operably coupled to the syringe. A pressure sensor may be positioned within a pathway of the inflation lumen configured to measure a pressure within the inflation lumen. A computer system may be operably coupled to the motorized inflation system. A display may be operably coupled to the computer system, the display configured to display a real-time pressure-volume curve based on the measured pressure and the measured volume. The computer system may be configured to identify at least four distinct phases of balloon inflation. A first phase of the at least four distinct phases of balloon inflation may correspond to initial balloon filling phase in which the measured pressure stays substantially constant as the measured volume increases, the pressure-volume curve having a first slope during the first phase of inflation. A second phase of the at least four distinct phases of balloon inflation may correspond to an additional balloon filling phase in which the measured pressure increases as the measured volume increases, the pressure-volume curve having a second slope during the second phase of inflation, the second slope being greater than the first slope. A third phase of the at least four distinct phases of balloon inflation may correspond to contact between the balloon and the previously-implanted prosthetic surgical valve in which the measured pressure increases as the measured volume increases, the pressure-volume curve having a third slope during the third phase of inflation, the third slope being greater than the second slope. A fourth phase of the at least four distinct phases of balloon inflation may correspond to a frame of the previously-implanted prosthetic surgical valve being fractured in which the measured pressure decreases as the measured volume increases. The computer system may be configured to instruct the motorized inflation system to stop advancing inflation media out of the syringe upon detecting the fourth phase of balloon inflation. The computer system may be configured to receive a critical balloon pressure threshold set point. The computer system may be configured to instruct the motorized inflation system to stop advancing inflation media out of the syringe upon detecting the measured pressure has reached the critical balloon pressure threshold set point.

According to an example of the disclosure, a balloon catheter system is configured to fracture a previously-implanted prosthetic heart valve, and the balloon catheter system includes a balloon catheter including a balloon at a distal end thereof, the balloon configured to be in fluid communication with a fluid reservoir via an inflation lumen. At least one stress concentrator may be coupled to an outer surface of the balloon, the at least one stress concentrator extending in a proximal-to-distal direction of the balloon. The balloon may have a length in the proximal-to-distal direction, and the at least one stress concentrator may extend at least 50% of the axial length of the balloon. Upon inflation of the balloon within the previously-implanted prosthetic heart valve, the at least one stress concentrator may be configured to contact the previously-implanted prosthetic heart valve prior to the outer surface of the balloon contacting the previously-implanted prosthetic heart valve. A pressure sensor may be positioned within a pathway of the inflation lumen and may be configured to measure a pressure within the inflation lumen. The at least one stress concentrator may have a leading contact surface having a cross-sectional shape selected from the group consisting of: semi-circular, triangular, and rectangular. The at least one stress concentrator may define a leading contact surface facing radially away from the outer surface of the balloon, the leading surface being configured to lacerate at least one of a native heart valve leaflet and a prosthetic leaflet of the previously-implanted prosthetic heart valve. The at least one stress concentrator may include three stress concentrators distributed at substantially equal intervals around a circumference of the balloon.

According to an example of the disclosure, a method of fracturing a previously-implanted prosthetic surgical heart valve includes advancing a non-compliant balloon of a balloon catheter through a patient's vasculature until the balloon is positioned at least partially within the previously-implanted prosthetic surgical heart valve. Inflation media may be advanced through an inflation lumen and into the balloon to inflate the balloon. At least one stress concentrator coupled to an outer surface of the balloon may be contacted with an interior surface of the previously-implanted prosthetic surgical heart valve as the balloon inflates, prior to the outer surface of the balloon contacting the previously-implanted prosthetic surgical heart valve. The method may include continuing to advance inflation media through the inflation lumen to further inflate the balloon until a frame of the previously-implanted prosthetic surgical heart valve fractures. The at least one stress concentrator may have a leading contact surface having a cross-sectional shape selected from the group consisting of: semi-circular, triangular, and rectangular. The at least one stress concentrator may define a leading contact surface facing radially away from the outer surface of the balloon, so that as the balloon further inflates, the at least one stress concentrator lacerates at least one of (i) a prosthetic leaflet of the previously-implanted surgical heart valve or (ii) a native leaflet a native heart valve of the patient. The at least one stress concentrator may include three stress concentrators distributed at substantially equal intervals around a circumference of the balloon. The method may include imaging the three stress concentrators prior to contacting the three stress concentrators with the interior surface of the previously-implanted prosthetic surgical heart valve. The balloon may be rotated to align the three stress concentrators with selected components of the previously-implanted prosthetic heart valve after imaging the three stress concentrators, but prior to contacting the three stress concentrators with the interior surface of the previously-implanted prosthetic surgical heart valve. The selected components of the previously-implanted prosthetic surgical heart valve may be commissure features of the frame of the previously-implanted prosthetic surgical heart valve. The selected components of the previously-implanted prosthetic surgical heart valve may be pre-formed weak areas within the frame of the previously-implanted prosthetic surgical heart valve. The pre-formed weak areas may be thinned areas of the frame of the previously-implanted prosthetic surgical heart valve. The pre-formed weak areas may be perforated areas of the frame of the previously-implanted prosthetic surgical heart valve.

According to an example of the disclosure, a method of performing a valve-in-valve implantation includes advancing a balloon of a balloon catheter through a patient's vasculature until the balloon is positioned at least partially within the previously-implanted prosthetic surgical heart valve, a balloon-expandable prosthetic heart valve being collapsed on the balloon during the advancing. Inflation media may be advanced through a first inflation lumen and into the balloon. During the advancing of the inflation media through the first inflation lumen, the method may include (i) covering a proximal portion of the balloon and the balloon-expandable prosthetic heart valve with a covering sheath such the proximal portion of the balloon and the balloon-expandable prosthetic heart valve are restricted from expanding, and (ii) uncovering a distal portion of the balloon with the covering sheath such that the distal portion of the balloon inflates and fractures a frame of the previously-implanted prosthetic surgical heart valve. After fracturing the frame of the previously-implanted prosthetic surgical heart valve, the covering sheath may be withdrawn to expose the balloon-expandable prosthetic heart valve and the proximal portion of the balloon. After withdrawing the covering sheath, inflation media may be advanced through a second inflation lumen to inflate the balloon and to expand the balloon-expandable prosthetic heart valve into the previously-implanted prosthetic surgical heart valve. The balloon may be a single balloon and the first inflation lumen and the second inflation may be the same. A pressure sensor may be positioned within a pathway of the first inflation lumen. The balloon may include a primary balloon including the proximal portion of the balloon and the distal portion of the balloon, and a secondary balloon may be positioned interior of the distal portion of the primary balloon. The first inflation lumen may be different than the second inflation lumen, the first inflation lumen leading to an interior of the secondary balloon, the second inflation lumen leading to an interior of the primary balloon. A first pressure sensor may be positioned within a pathway of the first inflation lumen, and a second pressure sensor may be positioned within a pathway of the second inflation lumen. The balloon may not be removed from the patient in a time period (i) after the frame is fractured and (ii) before the balloon-expandable prosthetic heart valve is expanded into the previously-implanted prosthetic surgical heart valve. The method may include repositioning the balloon-expandable prosthetic heart valve into alignment with the previously-implanted prosthetic surgical heart valve after fracturing the frame but before expanding the balloon-expandable prosthetic heart valve. Repositioning the balloon-expandable prosthetic heart valve into alignment with the previously-implanted prosthetic surgical heart valve may include (i) axially aligning the balloon-expandable prosthetic heart valve with the previously-implanted prosthetic surgical heart valve, and/or (ii) rotationally aligning commissures of the balloon-expandable prosthetic heart valve with commissures of the previously-implanted prosthetic surgical heart valve.

According to an examples of the disclosure, a balloon catheter system for a valve-in-valve replacement includes a balloon catheter including a balloon at a distal end thereof, the balloon catheter configured to be in fluid communication with a fluid reservoir via an inflation lumen. A balloon-expandable prosthetic heart valve may be configured to be maintained over a central portion of the balloon in a collapsed condition. A covering sheath may be axially moveable relative to the balloon between (i) a first condition in which the covering sheath covers the balloon-expandable prosthetic heart valve and a proximal portion of the balloon to restrict the balloon-expandable prosthetic heart valve and the proximal portion of the balloon from expanding, but does not cover a distal portion of the balloon so that the distal portion of the balloon is expandable, and (ii) a second condition in which the covering sheath does not cover the balloon-expandable prosthetic heart valve, the proximal portion of the balloon or the distal portion of the balloon. The balloon may be a single balloon, and an inflation lumen may be configured to fluidly connect the fluid reservoir to an interior volume of the balloon. A pressure sensor may be positioned within a pathway of the inflation lumen. The balloon may include (i) a primary balloon including the proximal portion of the balloon, the central portion of the balloon, and the distal portion of the balloon, and (ii) a secondary balloon positioned interior of the distal portion of the primary balloon. The inflation lumen may be a first inflation lumen and the fluid reservoir may be a first fluid reservoir. The first inflation lumen may be configured to fluidly connect the first fluid reservoir to an interior volume of the primary balloon, and a second inflation lumen may be configured to fluidly connect a second fluid reservoir to an interior volume of the secondary balloon. The first fluid reservoir and the second fluid reservoir may be different fluid reservoirs. A first pressure sensor may be positioned within a pathway of the first inflation lumen, and a second pressure sensor may be positioned within a pathway of the second inflation lumen.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the example of the handle of the delivery system shown in FIG. 4.

FIG. 6 is an enlarged view of an example of the distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the example of the inflation system of FIG. 8.

FIG. 10 is a perspective view of an example of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12A is a perspective view of an example of a surgical prosthetic heart valve.

FIG. 12B is perspective view of an example of a frame of the surgical prosthetic heart valve of FIG. 12A.

FIG. 12C is a perspective view of an example of a filler ring of the surgical prosthetic heart valve of FIG. 12A.

FIG. 12D is a perspective view of an example of an assembly that includes the frame of FIG. 12B and the filler ring of FIG. 12C covered inside and out by a fabric tube.

FIG. 13A is a schematic view of an example of a system for fracturing a previously-implanted surgical prosthetic heart valve.

FIG. 13B is an example of data being recorded by and/or displayed on the example system of FIG. 13A.

FIG. 13C is a flowchart providing an example method of use relating to the configuration(s) of FIGS. 13A-13B.

FIG. 14A is a top view of an example of a balloon, of a balloon catheter, with stress concentrators coupled to the balloon.

FIGS. 14B-14D are examples of shapes that may be used for the stress concentrators of FIG. 14A.

FIG. 14E is a flowchart providing an example method of use relating to the configuration(s) of FIGS. 14A-14D.

FIG. 15A shows an example of a distal end of a delivery system configured to crack a prosthetic surgical valve and to deliver a new prosthetic heart valve in a valve-in-valve procedure.

FIG. 15B shows another example of a distal end of a delivery system configured to crack a prosthetic surgical valve and to deliver a new prosthetic heart valve in a valve-in-valve procedure.

FIG. 15C is a flowchart providing an example method of use relating to the configuration(s) of FIGS. 15A-15B.

FIG. 16 is a block diagram that illustrates an example of a computer system upon which an example may be implemented.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

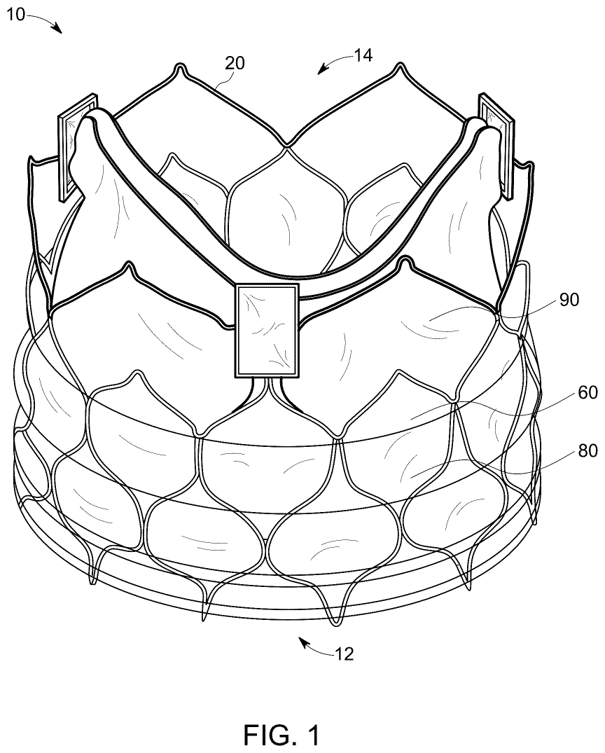

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

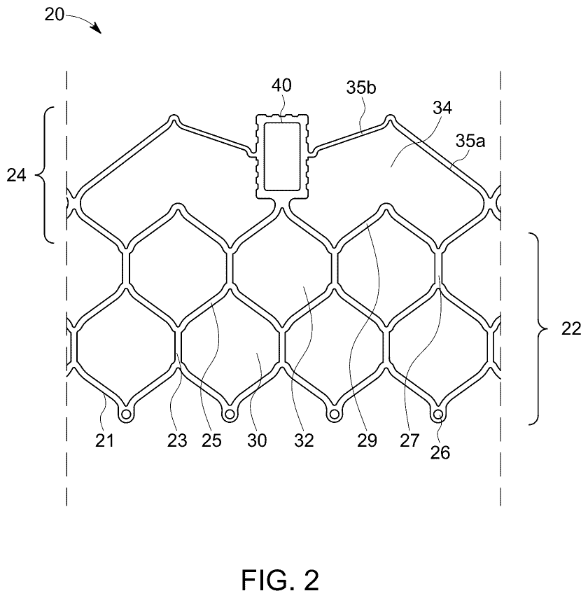

Now referring in addition to FIG. 2., FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

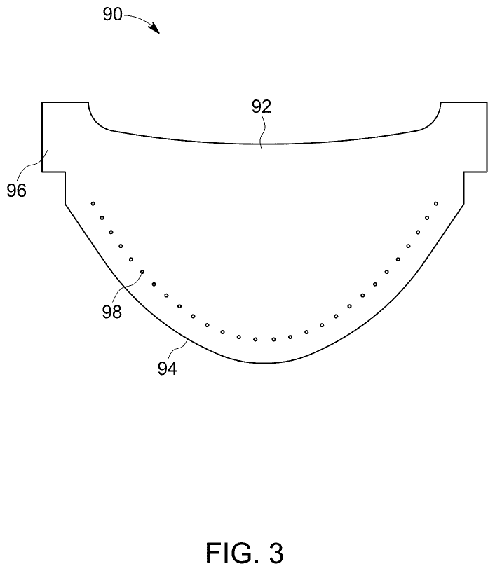

Now referring in addition to FIG. 3., FIG. 3 is a front view of an example of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

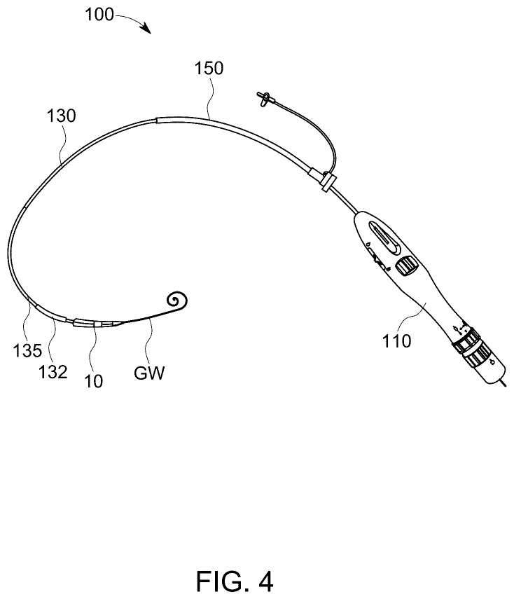

Now referring in addition to FIG. 4, FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introductory of 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

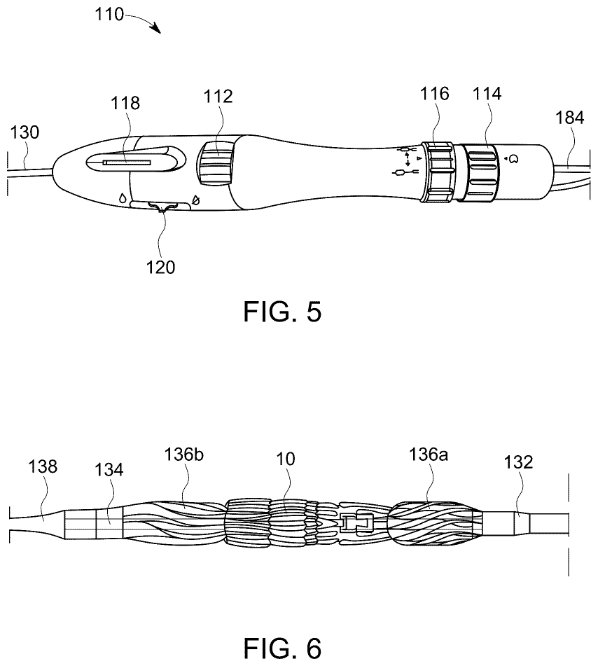

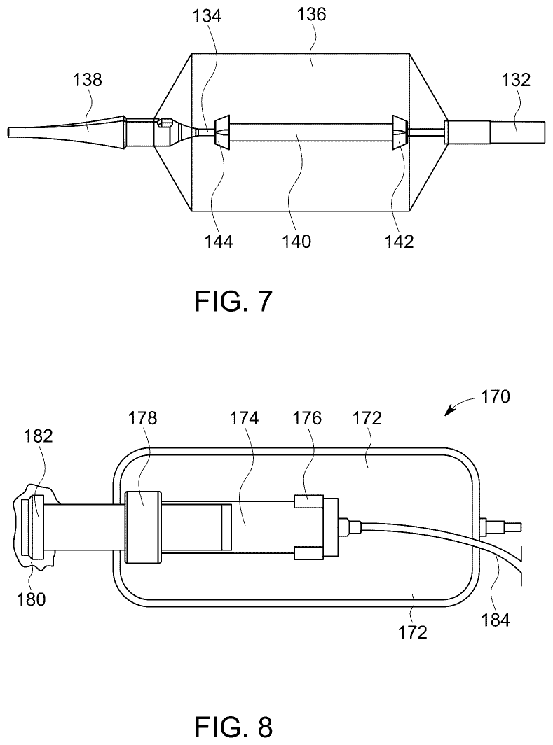

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g. the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135. In some examples, the handle may include a window 118 that allows viewing of an indicator that corresponds to the amount of catheter deflection. For example, a carrier to which the indicator is attached may be attached to the steering wire. In some examples, when there is minimum (or zero) tension on the steering wire, the indicator is at the far distal position within window 118, but as deflection is actuated, for example by drawing a carrier proximally (and tensioning the steering wire as the carrier draws proximally), the indicator will move proximally along window 118, giving the user a readily-apparent indication of the amount of deflection applied to the catheter at any given moment.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

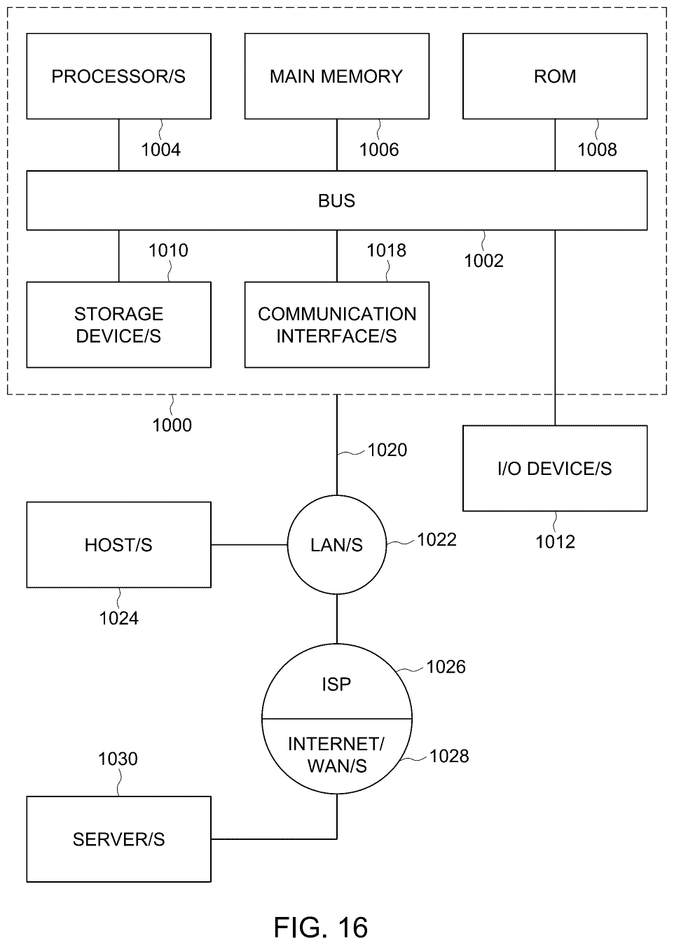

In addition to steering and positioning actuators, delivery system 100 may include a balloon actuator 120. Balloon actuator 120 may be an input device, for example similar to those described in connection with FIG. 16, that sends instructions to inflation system 170 or another device configured to control inflation. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In some examples, balloon 136 may be a semi-compliant balloon, for example formed from a polyether block amide offered under the tradename Pebax®, including, e.g., 74D Pebax. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Now referring in addition to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

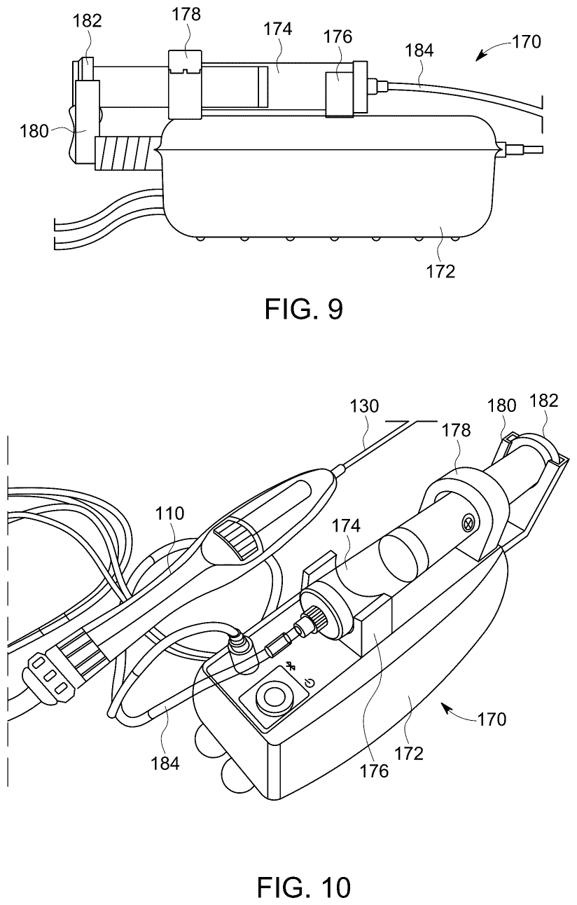

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. In some embodiments, the inflation system 170 forms a computer system, or components thereof, such as shown and described in connection with FIG. 16. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in the examples of each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

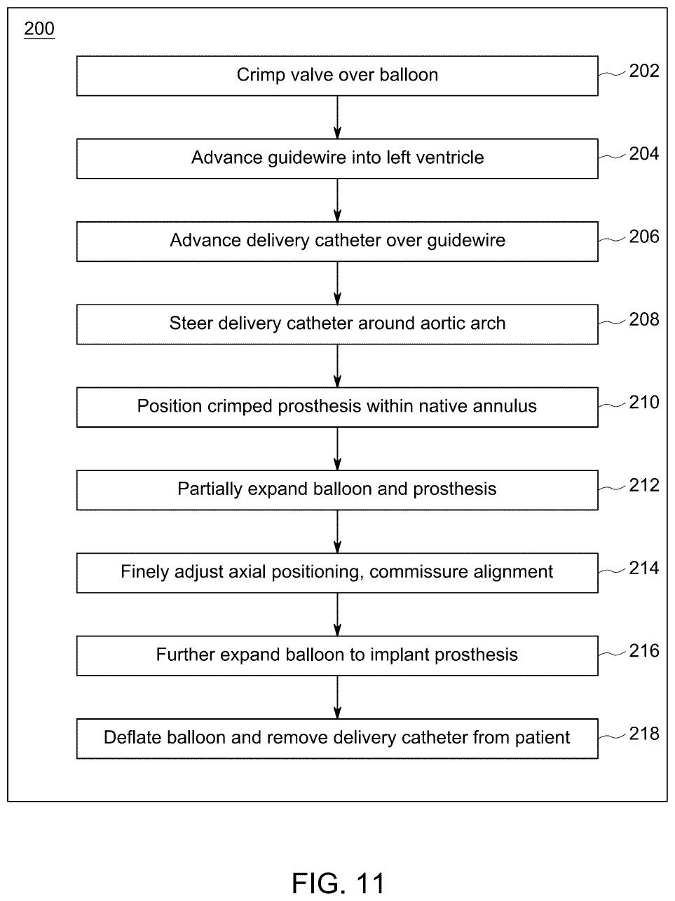

Now referring in addition to FIG. 11, FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieve and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

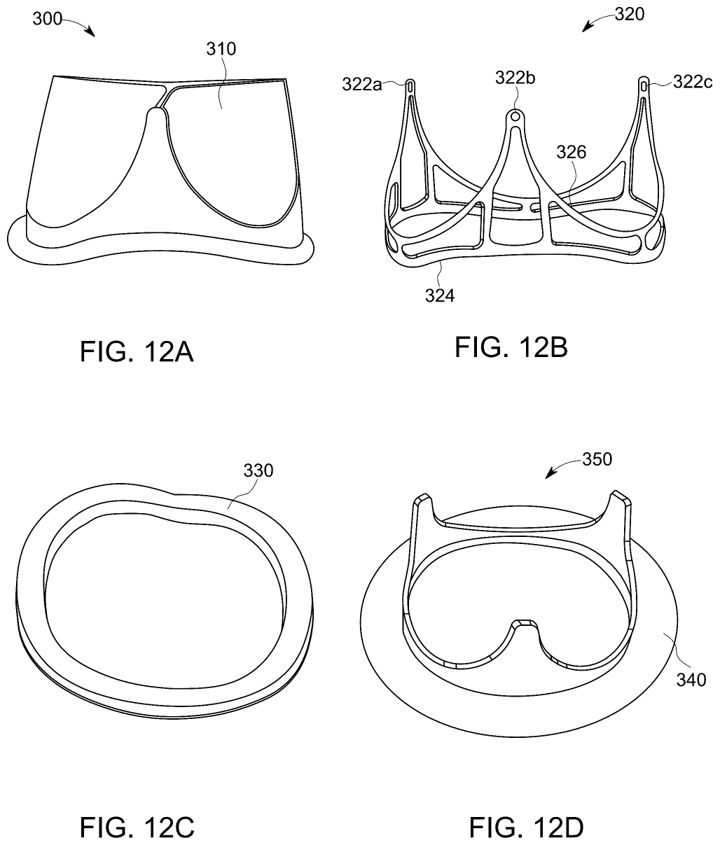

Referring now in addition to FIG. 12A, FIG. 12A shows an example of a surgical prosthetic heart valve 300, although it should be understood that surgical prosthetic heart valves may take many other forms. In the illustrated example, surgical valve 300 includes three bioprosthetic tissue leaflets 310, that are mounted to a frame 320, and example of which is shown in FIG. 12B. Frame 320 in the illustrated example is a hollow, annular, stent-like structure (sometimes referred to simply as a stent). Frame 320 may be referred to as “hollow” because the interior that is bounded by its annular structure is open. Frame 320 in some examples is made of metal such as titanium. An exemplary technique for making frame 320 is to cut it from a tube using a laser, and then electro-polishing the frame 320. In some examples, frame 320 has three commissure portions or regions 322a, 322b, and 322c that are equally spaced from one another around the circumference of the stent. In this example, each commissure portion 322a-322c stands up from the annularly continuous base portion of frame 320. The base portion in some examples includes a lower-most, inflow edge portion 324. In the illustrated example, inflow edge portion 324 may be scalloped as one proceeds around frame 320 to approximately match the natural scallop of the native valve annulus. For example, this scallop may rise in the vicinity of each commissure region 322a-322c, and it may fall between each annularly adjacent pair of commissures. It should be understood that frame 320 may include three commissure regions 322a-322c to accommodate three prosthetic heart valve leaflets 310, although in other embodiments, the frame may include more or fewer commissure regions, for example to accommodate a two-leaflet valve or a four-leaflet valve. Further, it should be understood that frame 320 may be adapted for use in a surgical prosthetic heart valve intended to replace a native aortic valve or another native heart valve.

Still referring to FIG. 12A, inflow edge 324, outflow edge 326, and the flexibility of frame 320 may in some examples be designed to help ensure proper opening and coaptation of the finished surgical prosthetic heart valve (e.g. prosthetic surgical heart valve 300) in use. Frame 320 may in some examples be further designed to decrease maximum stresses in use, which may give the finished valve an increased safety factor. Although titanium is mentioned above as an example of a material from which frame 320 can be made, other materials are also possible, including for example Elgiloy MP35N, or polymers such as PEEK or acetal.

Referring now in addition to FIG. 12C, FIG. 12C is a perspective view of an example of a filler ring 330, which in some examples may be formed of silicone. Referring in addition to FIG. 12D, FIG. 12D illustrates an example of an assembly 350 that includes stent frame 320 and filler ring 330 covered inside and out by fabric tube 340. For example, frame 320 and ring 330 may be placed coaxially around the outside of a lower portion of fabric tube 340, and ring 330 may be located outside inflow edge portion 324. In some examples, the upper portion of fabric 340 may then be pulled down over the outside of frame 320 and ring 330 and pulled tightly enough to conform to outflow edge portion 324 as shown in the example of FIG. 12D. Sutures may be used, in one example, to hold the above-described components together in the condition shown in FIG. 12D. In use, the surgical prosthetic heart valve 300 may be surgically implanted into a patient to replace a native heart valve, such as a native aortic valve, by gaining surgical access to the heart and suturing the prosthetic heart valve 300 into the native heart valve annulus by passing sutures through the ring 330 and the native tissue. It should be understood that the surgical prosthetic heart valve 300 and exemplary components shown and described in connection with FIGS. 12A-D are just one example of a surgical heart valve, which is described in more detail in U.S. Pat. No. 8,353,954, the disclosure of which is hereby incorporated by reference herein.

After a surgical prosthetic heart valve (e.g., surgical prosthetic heart valve 300) has been implanted into a patient (e.g., to replace a patient's native aortic valve), the prosthesis may begin to fail over time. In one example, years or decades after the surgical prosthetic heart valve is implanted, bioprosthetic tissue leaflets (e.g., leaflets 310) may begin to calcify and the prosthesis may become inefficient at controlling blood flow through the prosthesis. In such examples, it may be desirable to replace the original prosthetic heart valve with a new prosthetic heart valve, such as a collapsible and expandable prosthetic heart valve (e.g., prosthetic heart valve 10). However, one primary impediment to such a secondary valve implantation, which may be referred to as a “valve-in-valve” procedure or implantation, is that, by nesting a newly-implanted prosthetic heart valve within a previously-implanted prosthetic heart valve, there tends to be a significantly reduced the cross-sectional area for blood flow due at least in part to the structure of the previously-implanted prosthetic heart valve. This reduction in area for blood flow in some examples results in undesirable increases in pressure gradients across the newly-implanted prosthetic heart valve, and similarly may result in a decrease effective orifice area (“EOA”). One approach to overcoming this potential problem is that the previously-implanted prosthetic surgical heart valve may be “cracked” prior to implanting the new prosthetic heart valve. As used here, the terms “cracking” and “fracturing” refer to breaking the otherwise continuous circumferential structure formed by the previously-implanted prosthetic surgical heart valve, for example the frame 320 of FIG. 12B, and for example in particular the inflow edge portion 324. As an example, the inflow edge portion 324 of frame 320 may provide a maximum possible size to which a newly-implanted prosthetic heart valve may be expanded when the inflow edge portion 324 remains intact. However, if the frame 320, and in particular the inflow edge portion 324, is cracked so that it is no longer circumferentially continuous, a newly implanted prosthetic heart valve may be able to be expanded to a larger size, not limited (or not as limited) by the existing structure of the previously-implanted prosthetic surgical heart valve. In some examples, this “cracking” or fracturing may be achieved by inflating a balloon within the previously-implanted prosthetic surgical heart valve, including for example a non-compliant balloon to fracture the frame of the prosthetic surgical heart valve. As should be understood from the above, such cracking or fracturing of the frame of the previously-implanted prosthetic surgical heart valve may allow a secondary prosthetic heart valve to be implanted within the previously-implanted prosthetic surgical heart valve, and expanded to a larger diameter than may have been possible in the absence of performing the cracking or fracturing. This, in turn, may help to minimize or eliminate impairment of blood flow through the newly implanted prosthetic heart valve.

In some examples, if a previously-implanted prosthetic heart valve is being cracked or fractured prior to implantation of a new expandable prosthetic heart valve (including self-expandable, mechanically-expandable, or balloon-expandable heart valves), it may be desirable to have confirmation that the cracking or fracturing is successful before the new prosthetic heart valve is fully implanted. In some examples, if a user keeps inflating a balloon into a previously-implanted prosthetic heart valve to fracture the previously-implanted prosthetic heart valve, a risk of annulus rupture or a burst balloon may increase if the balloon is inflated too much. This situation may occur if the previously-implanted prosthetic heart valve is actually fractured but the user does not realize it has been fractured. In some situations, if a non-compliant balloon is being used to crack the surgical prosthetic heart valve, an audible popping will be heard and/or tactile feedback may occur (e.g., via an inflation lumen) to provide some level of indication that the cracking has been successful. However, that type of feedback does not always occur, and in any event such feedback relies largely on a subjective determination by the physician or other personnel. Thus, it would be desirable to have an objective determination that cracking of the prosthetic surgical heart valve has been achieved prior to completing the new prosthetic heart valve implantation.

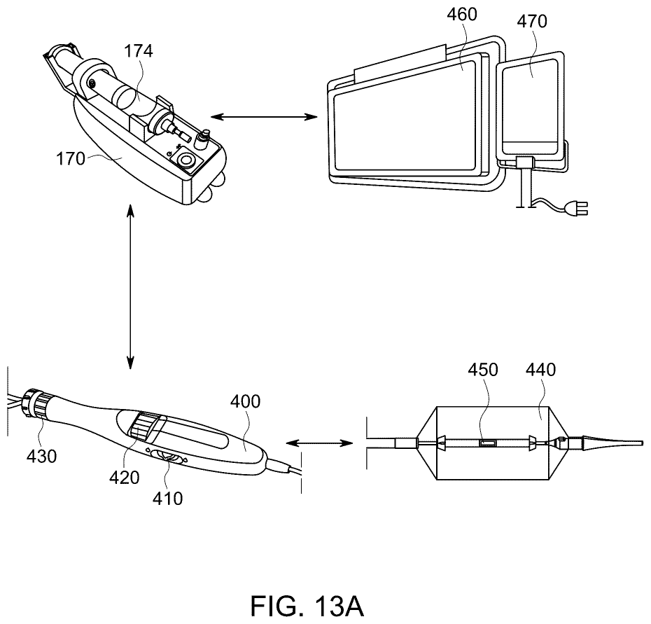

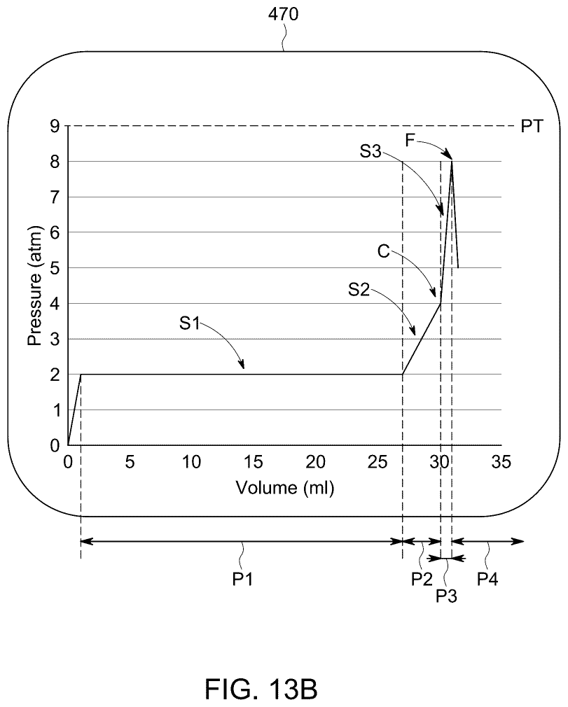

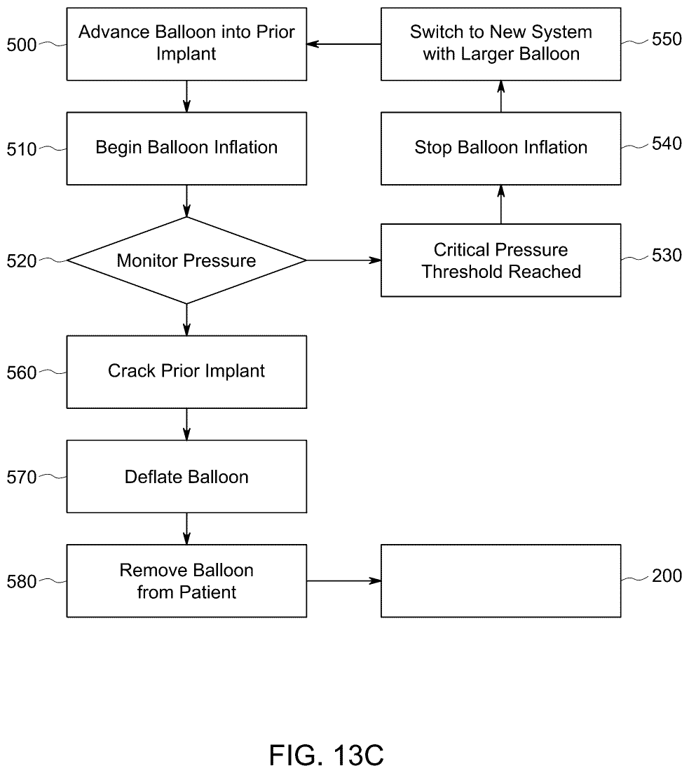

Referring now in addition to FIG. 13A, FIG. 13A is a schematic view of an example of a system for fracturing a previously-implanted surgical prosthetic heart valve. In the illustrated example, the system includes a balloon inflation system, which in some examples is the same as the balloon inflation system 170 described above, including a syringe 174 or other inflation media reservoir operatively coupled to the balloon inflation system 170. Because the balloon inflation system may be the same as balloon inflation system 170, it is not described again in detail here. In some example, the balloon inflation system 170 may be operatively coupled to a balloon catheter handle 400 (e.g., via a fluid line and cables that transmit power and/or data) in the same or a similar way that the balloon inflation system 170 is coupled to handle 110 as described above. In some examples, the balloon catheter handle 400 may be similar or the same as handle 110, and include for example a balloon actuator 410 (which may be similar or identical to balloon actuator 120), a steering knob 420 (which may be similar or identical to steering knob 112), and an axial alignment actuator 430 (which may be similar or identical to axial alignment actuator 116). It should be understood that, although in some embodiments the balloon catheter handle 400 is similar or identical to handle 110, in other examples there may be differences, for example one or more of the steering knob 420 and axial alignment actuator 430 may be omitted.

Still referring to FIG. 13A, the balloon catheter handle 400 may be operatively coupled to an inflatable balloon 440 (e.g., via one or more catheter lines which may be similar to those described in connection with delivery system 100). In some examples, balloon 440 may be a non-compliant balloon in order to better apply outward force during inflation, for example to a surgical prosthetic heart valve. In some examples, the non-compliant balloon may be formed of a material such as nylon (polyamide) 12 or other polyamides, including those offered under the tradename Vestamid® Care ML21 or Grilamid®. The structure of balloon 440 and related components may, in some examples, be similar or identical to that of balloon 136 and related components. However, in some examples, the structure is different, for example the balloon 440 may omit the pillowed proximal balloon portion 136a and the pillowed distal balloon portion 136b shown and described in connection with FIG. 6. A sensor 450, which in some examples is a pressure sensor, may be positioned anywhere within the path of the inflation lumen that extends between syringe 174 and the interior of the balloon 440. In the illustrated example, the pressure sensor 450 is mounted to an internal shaft within the balloon 440, but it should be understood that this is only one exemplary position. The pressure sensor 440 is operatively coupled to the balloon inflation system 170 so that data (e.g., pressure readings) may be transmitted from the pressure sensor 450 to the balloon inflation system 170.

Still referring to FIG. 13A, similar to the inflation system 170 described in connection with delivery system 100, the inflation system 170 operably coupled to balloon catheter handle 400 may in some examples also be operably coupled to a computer 460 (which may have an integrated display) and/or to a mobile display 470, such as a tablet. Computer 460 and tablet 470 may in some examples individually, in combination, or along with other components, form a computer system (or a portion thereof) as described in connection with FIG. 16. The data connections between the balloon inflation system 170 and the balloon catheter handle 400 and/or computer 460 may be wired or wireless. In some examples, the computer 460 may receive real-time readings from pressure sensor 450 (which may be relayed through inflation system 170), and those real-time readings may be graphically displayed on the computer 460 and/or on an associated tablet 470. Similarly, in some examples, data regarding the state of inflation of balloon 440, including for example volume of inflation media passed into the balloon 440, may be displayed on the computer 460 and/or on an associated tablet 470. For example, due at least in part to the motorized driving of syringe 174 by inflation system 170, the inflation system 170 may know at any point how much inflation media has been passed from the syringe 174 to the balloon 440 (or vice versa), which information may be transmitted to the computer 460 for display along with the pressure data.

Referring now in addition to FIG. 13B, FIG. 13B is an example of data being transmitted to the computer 460 and displayed on the computer 460 and/or tablet 470 for review by the physician or other user during an example procedure. If balloon 440 is being used to crack a previously-implanted prosthetic surgical heart valve, a real-time display of pressure (e.g., the pressure reading of pressure sensor 450) versus volume (e.g., the amount of inflation media that the inflation system 170 has pushed to the balloon 440) may be provided (e.g., on the tablet 470) so that objective data relating to the surgical valve cracking may be reviewed. In some examples, up to four or more distinct phases of inflation of the balloon 440 may be identified by reviewing the pressure-volume data being displayed in real time. For example, during a first main phase P1 of balloon inflation, the balloon 440 begins to fill with inflation media, and the pressure reading remains substantially constant (e.g., +/−10% deviation from a horizontal slope S1) as the balloon 440 fills. As the balloon 440 continues to inflate, but before contact is made with the prosthetic surgical heart valve, a normal balloon compliance phase P2 may be expected in which the pressure begins to increase as volume increases, for example at a second slope S2 greater than the first slope S1. This second phase P2 may continue until enough volume has passed into the balloon 440 to increase its size so that it contacts an interior surface of the surgical prosthetic heart valve (e.g., surgical valve 300). At this point of contact C, a third phase P3 may begin in which pressure increases more rapidly for each unit of additional volume of inflation media passed into the balloon 440, resulting in a third slope S3 that is greater than the second slope S2. When the transition between slope S2 and S3 occurs, and thus the procedure moves from the second phase P2 to the third phase P3, the operator may become aware that contact with, but no cracking/fracture of, the surgical prosthetic heart valve has occurred. As the balloon 440 continues to inflate and apply pressure to the previously-implanted prosthetic surgical heart valve, eventually enough force is applied to the prosthetic surgical heart valve to crack or fracture it (e.g., to fracture the circumferential continuity of the frame). Immediately beyond this point of fracture F, the pressure may drop rapidly because the frame (e.g., frame 320) of the prosthetic surgical heart valve is providing significantly less resistance to the inflation. Once this fourth phase P4 is achieved, in which pressure rapidly begins to drop despite inflation continuing, the user may have objective evidence that the prosthetic surgical heart valve has been successfully cracked or fractured. In some examples, during the third phase P3, inflation of the balloon 440 may be at a substantially constant rate (e.g. volumetric flow of fluid into the balloon 440 may be substantially constant over time). In some examples, during the third phase P3, inflation of the balloon 440 may be at a non-constant rate (e.g. volumetric flow of fluid into the balloon 440 may change, including the flow rate decreasing over time as the fracture becomes imminent). In still other examples, during the third phase P3, the system may alternate between pushing fluid into the balloon 440 and withdrawing fluid from the balloon 440, for example inducing pulsations into the balloon 440 as the fracture becomes imminent.

Referring still to FIG. 13B, after the fracture point F occurs and the fourth phase P4 is entered, the physician (or other user) may stop inflating the balloon 440. In some examples, it may be desirable to stop the inflation very quickly after fracture has occurred, because continued inflation could result in applying enough force to the native valve annulus to rupture the native valve annulus, which is undesirable. In some examples, the physician may manually stop inflation after the fracture point F, for example by releasing pressure being applied to the balloon actuator 410. However, in other examples, the inflation system 170 may be set to automatically stop inflating the balloon 440 upon detecting a rapid drop in pressure readings despite increasing inflation volume being passed to the balloon 440. In some examples, the inflation system 170 may be programmed to discontinue inflation, regardless of whether the user is applying force to the balloon actuator 410, upon a significant (e.g., 10% or more, 15% or more, 20% or more, 25% or more, etc.) drop in pressure with continued increase in inflation volume. To provide complete (or substantially complete) control to the user, in some examples, the user may be able to restart inflation, if the user believes it appropriate to do so after the automatic shutoff, by reengaging the balloon actuator 410. In other words, in addition to providing objective evidence of valve cracking, in some examples the pressure-volume data may be used, manually and/or automatically, as a safety feature to prevent rupturing the native annulus, or bursting the balloon 440, via overinflation of the balloon 440. In some examples, if the previously-implanted prosthetic heart valve has already fractured, it may present a sharp edge that could increase the risk of bursting the balloon 440 if balloon inflation continues after fracturing is achieved.

While annulus rupture is one safety concern when performing surgical valve cracking with a non-compliant balloon, another safety concern is bursting the balloon. In the case of a burst balloon, the outcomes can range from, for example, user inconvenience from needing to get a new balloon inserted into the patient, to saline-contrast solution that erupts from the balloon traveling to the patient's kidneys and causing kidney failure. Other complications involve adverse reactions to increased procedure time. Bursting the balloon may be a particular concern when the size of the balloon 440 selected for the procedure is too small relative to the size of the annulus and/or relative to the size of the previously-implanted prosthetic surgical valve. In other words, if the balloon 440 size is too small, it may be prone to rupturing before it gets large enough to contact the prosthetic surgical valve and/or before it gets large enough to fracture the prosthetic surgical valve. In some examples, in order to avoid this risk of rupturing the balloon 440, a critical pressure threshold PT may be set. For example, FIG. 13B shows a pressure threshold PT of about 9 atm, which is an arbitrary value used here for purposes of illustration. In the example procedural chart of FIG. 13B, the critical pressure threshold PT is never reached. However, if the patient's valve annulus was larger than originally expected, and the balloon 440 kept on inflating until a pressure of about 9 atm without achieving the fracture point F, the inflation system 170 may be set to automatically stop the inflation. In other words, the critical pressure threshold PT may be set to a pressure value at which the balloon 440 has an unreasonably high risk of rupturing. In examples in which a critical pressure threshold PT is set, there may be little or no risk that the balloon 440 ruptures due to overinflation because, as soon as the critical pressure threshold PT is reached, the inflation system 170 aborts the inflation procedure. If determined appropriate by the user, in some examples, the user may choose to try the procedure again but with a larger-sized balloon 440 if the procedure is aborted following reaching the critical pressure threshold PT. In some examples, the critical pressure threshold PT may be illustrated on the chart and/or on the computer 460 or tablet 470 to provide the user with a visual indication of the amount of current balloon pressure compared to the critical pressure threshold PT, but in other examples such a visual indicator may be omitted. As with other safety features provided herein, the user may be provided in some examples with the option of overriding the safety feature and continuing inflation by reengaging the balloon actuator 410 if it is determined safe to do so.

Now referring in addition to FIG. 13C, FIG. 13C is a flowchart providing an example method of use relating to the configuration(s) of FIGS. 13A-13B. As shown in FIG. 13C, in a first exemplary step 500, a user may advance the balloon 440 of the balloon catheter (which may include handle 400 attached to inflation system 170) into a patient and at least partially inside a previously-implanted prosthetic surgical heart valve (e.g., valve 300 of FIG. 10A) while the balloon 440 is in a deflated condition. Once the deflated balloon 440 is within the prosthetic surgical heart valve, in exemplary step 510, the user may initiate a balloon inflation process, for example by pressing and holding balloon actuator 410 to signal inflation system 170 to actuate its motor to depress syringe 174 to force fluid (e.g., saline) through the handle 400 and into the balloon 440. During step 520, the user may monitor (e.g., by viewing real-time data being charted on tablet 470 similar to that shown in FIG. 13B) pressure within the inflation line compared to the volume of inflation media pass to the balloon 440. In some examples, a critical pressure threshold PT may be set, and step 530 represents the scenario in which the critical pressure threshold PT is achieved before cracking or fracturing of the surgical prosthetic heart valve is achieved. If step 530 is reached, in step 540 the inflation of balloon 440 may be discontinued, for example either automatically by the inflation system 170, or manually by the user taking pressure off the balloon actuator 410. In step 550, the balloon 440 may be deflated and removed from the patient and a larger balloon size chosen. After the larger balloon size is chosen, the process may begin again at step 500 in another attempt at cracking the surgical prosthetic heart valve. In some examples, whether or not a critical pressure threshold PT is set, inflation of the balloon 440 may continue until cracking of the prosthetic surgical heart valve is achieved, as represented by step 560. Cracking of the prosthetic surgical heart valve may be detected, for example, by manual viewing of the pressure-volume curve and recognizing a rapid pressure drop at the end of the third stage S3 of inflation, or automatically by the inflation system 170 upon detecting a threshold level of pressure drop as volume continues to increase. After cracking of the surgical prosthetic heart valve is detected, inflation of the balloon 440 may stop at step 570, for example either automatically by the inflation system 170, or manually by the user taking pressure off the balloon actuator 410, to avoid increased risk of rupturing the patient's annulus. After inflation of the balloon 440 stops at step 570, the balloon 440 may be actively deflated, for example by pressing the balloon actuator 410 in the opposite direction, and the balloon catheter may be removed from the patient in step 580. After the balloon catheter is removed from the patient, an expandable prosthetic heart valve may be implanted into the cracked surgical prosthetic heart valve. In one example, method 200 (e.g., as shown and described in connection with FIG. 11) may be performed to achieve the valve implantation, with the exception that method 200 is performed as a valve-in-valve implantation. However, it should be understood that any other transcatheter valve replacement (including self-expandable or balloon-expandable valves, for example) may be suitable for the valve-in-valve implantation following cracking of the surgical prosthetic heart valve.

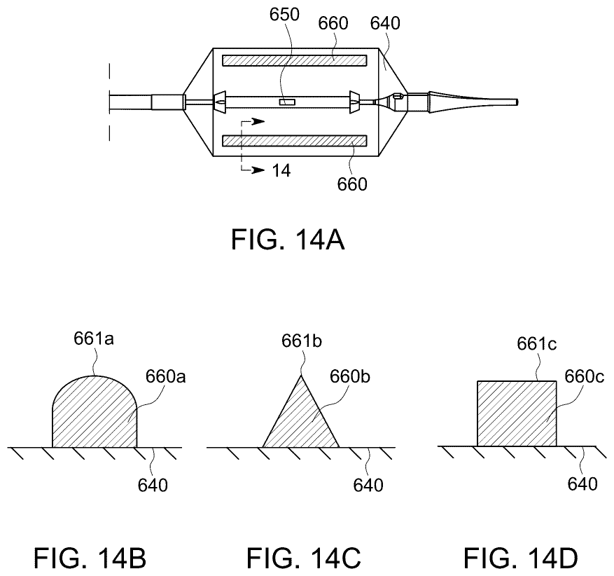

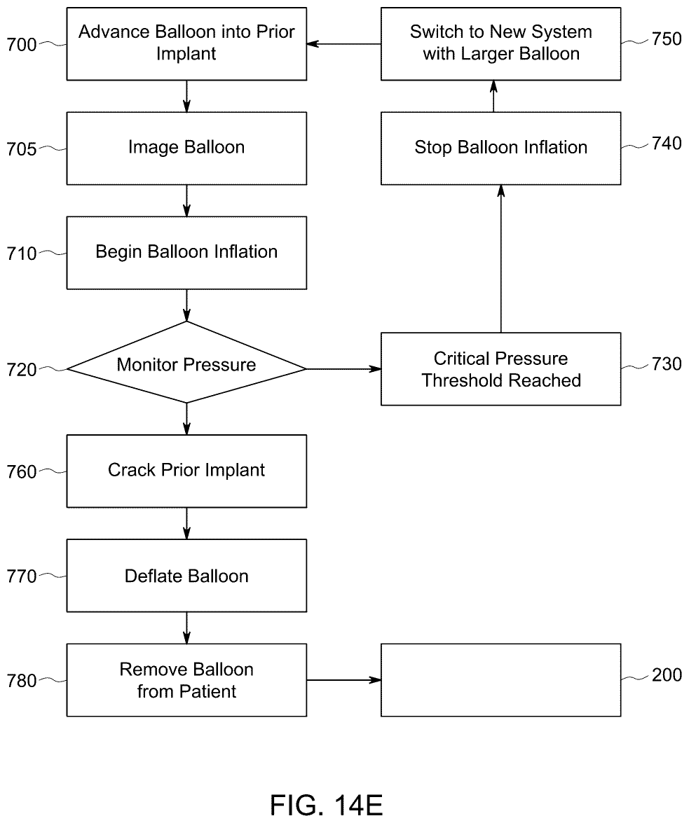

Now referring in addition to FIG. 14A, FIG. 14A is a top view of an example of a balloon 640 that may be a non-compliant balloon configured for cracking or fracturing a previously-implanted prosthetic surgical heart valve (e.g., valve 300). It should be understood that balloon 640, and features described therewith, may be configured for use as a standalone balloon catheter device, or in place of balloon 440 shown and described in connection with FIG. 13A. Thus, for any features not explicitly described in connection with balloon 640 (including example accessory devices and methods of use), it should be understood that the description provided in connection with balloon 440 may be applicable to balloon 640. In some examples, a sensor, such as a pressure sensor 650, may be provided anywhere within the fluid-flow line, including as shown in the illustrated example within the interior space of the balloon 640. Pressure sensor 650 (and its use in connection with balloon 640) may be similar or identical to pressure sensor 450 (and its use in connection with balloon 440) and is thus not described again in detail here.

Whereas in some examples balloon 440 is configured to fracture or crack a previously-implanted prosthetic surgical valve using balloon pressure and direct contact between an outer surface of the balloon and the prosthetic surgical valve, balloon 640 is provided in some examples with one or more stress concentrators 660 to directly contact the prosthetic surgical valve during the cracking or fracturing process. The stress concentrator(s) 660 in some examples may be affixed to an outer surface of the balloon 640, for example via adhesives, and during a cracking procedure, contact between the stress concentrator(s) 660 and the prosthetic surgical heart valve may and result in locally increased stresses on the surgical valve frame to facilitate the fracture at lower balloon pressures than may otherwise be needed in the absence of the stress concentrator(s) 660. The ability to fracture the prosthetic surgical heart valve at lower balloon pressure may reduce the risk of annular rupture once the prosthetic surgical heart valve does crack. In some examples, the stress concentrators 660 may be formed of a material in the same polymer family as the balloon 640 (e.g., an amide). In these examples, the stress concentrators 660 may be chemically bonded to the balloon 640 during the process of blow molding the balloon 640. As one particular example, injection molded stress concentrators 660 formed of polyether block amide such as Pebax® could be seated into the outer diameter of a blow mold, and attached to the balloon when the parison is blown outward into the stress concentrators 660 when blow molding the balloon 640.

In some examples, the balloon 440 may include a single stress concentrator 660, two stress concentrators 660, three stress concentrators 660, or more. In some examples, the one or more stress concentrators 660 may each extend along an axial direction (e.g., in a proximal-to-distal direction and/or substantially parallel to a central longitudinal axis of the balloon 640). For example, in some examples the one or more stress concentrators 660 may each extend at least 25%, at least 50%, or at least 75% or more of an axial length of the balloon 640. The one or more stress concentrators 660 are preferably formed from a rigid material, including plastics or metals (or metal alloys). If more than one stress concentrator 660 is provided, they may be in some examples provided at substantially equal intervals around the circumference of the balloon 640 (e.g. 180 degrees +/−10 degrees if two are provided, 120 degrees +/−10 degrees if three are provided, etc.). The one or more stress concentrators 660 may have any suitable geometry. In one example, as shown in FIG. 14B, a cross-sectional shape of the stress concentrator 660a (e.g., taken along the section line 14 of FIG. 14A) may include a leading contact surface 661a that is generally semicircular so that a rounded surface faces outwardly (e.g., radially outward from the outer surface of the balloon 640). In another example, as shown in FIG. 14C, a cross-sectional shape of the leading contact surface 661b of the stress concentrator 660b (e.g., taken along the section line 14 of FIG. 14A) may be generally triangular so that a pointed or sharp surface faces outwardly (e.g., radially outward from the outer surface of the balloon 640). In a further example, as shown in FIG. 14D, a cross-sectional shape of the leading contact surface 661c of the stress concentrator 660c (e.g., taken along the section line 14 of FIG. 14A) may be generally rectangular or square so that a flat surface faces outwardly (e.g., radially outward from the outer surface of the balloon 640).

In some examples, the stress concentrators 660 may be provided in a number and orientation on or around the balloon 640 so that they can be aligned to specifically desired features on the prosthetic surgical valve so that force from the stress concentrators 660 (upon inflation of the balloon 640) may target specific desired areas of the prosthetic surgical valve. In some examples, the one or more stress concentrators 660 may be radiopaque so that they are visible under fluoroscopy, or have other properties detectable under a desired imaging modality, which may help with achieving a desired alignment between the stress concentrator(s) 660 and the prosthetic surgical heart valve. As one example, three stress concentrators 660 may be positioned on the balloon 640 at approximately 120 degree (+/−10 degree) intervals so that, upon inflation of the balloon 640, the three stress concentrators 660 may be aligned with commissure features (e.g., commissure portions or regions 322a, 322b, 322c of frame 320) of the prosthetic surgical heart valve. As another example, three stress concentrators 660 may be positioned on the balloon 640 at approximately 120 degree (+/−10 degree) intervals so that, upon inflation of the balloon 640, the three stress concentrators 660 may be aligned with the center of three prosthetic leaflets (e.g., prosthetic leaflets 310) of the prosthetic surgical heart valve. In other examples, the previously-implanted surgical prosthetic heart valve may have been designed with weak points (e.g., perforated or thinned sections of frame 320) specifically to facilitate future cracking or fracturing, and the stress concentrators 660 may be provided in a number and orientation configured to align with the pre-formed weak points of the prosthetic surgical heart valve.

Still referring to FIGS. 14A-14D, in some examples, the stress concentrator(s) 660 may be provided with a sharp end facing radially outwardly, including for example a tip similar to stress concentrator 660b. In such examples, the sharp end may be sharp enough to cut or otherwise lacerate or tear the prosthetic leaflets of the surgical prosthetic heart valve and/or the native leaflets of the native heart valve. In such examples, by cutting the native and/or prosthetic leaflets prior to implanting the new prosthetic heart valve, the likelihood of the native and/or old prosthetic leaflets obstructing the coronary arteries may be reduced, similar to a BASILICA (bioprosthetic or native aortic scallop intentional laceration to prevent iatrogenic coronary artery obstruction) type of procedure. In some examples, a first group of stress concentrators (e.g., stress concentrators 660b) may be provided to target the native and/or prosthetic leaflets during the cracking procedure, while a second group of stress concentrators (e.g., stress concentrators 660a or 660c) may be provided to target certain areas of the frame of the surgical heart valve (e.g., the commissure features). In one example of that type of configuration, a total of six stress concentrators 660 may be provided at intervals of about 60 degrees (+/−5 degrees) around the circumference of the balloon 640, alternating between stress concentrators 660 with a sharp cutting edge (e.g., stress concentrators 660b) and stress concentrators 660 configured to crack the prosthetic surgical heart valve (e.g., stress concentrators 660a, 660b, or 660c).

Now referring in addition to FIG. 14E, FIG. 14E is a flowchart providing an example method of use relating to the configuration(s) of FIGS. 14A-14D. As shown in FIG. 14E, in a first exemplary step 700, a user may advance the balloon 740 of the balloon catheter (which may include a handle similar or identical to handle 400 attached to an inflation system similar or identical to inflation system 170) into a patient and at least partially inside a previously-implanted prosthetic surgical heart valve (e.g., valve 300 of FIG. 10A) while the balloon 640 is in a deflated condition. Once the deflated balloon 640 is within the prosthetic surgical heart valve, in step 705, the balloon 640 may be imaged (e.g., under fluoroscopy) to determine if the one or more stress concentrators 660 are in a desired position relative to the prosthetic surgical heart valve and/or the native heart valve, and the position may be adjusted (e.g., rotationally be using a steering mechanism similar to commissure alignment actuator 114 and/or axially by using a steering mechanism similar to axial alignment actuator 116 or 430) to a desired position. It should be understood that imaging step 705, if performed, may be performed at more than one stage and at different stages during the procedure as desired. After confirming the desired position and/or orientation of the stress concentrator(s) 660 (if such a step is performed), in exemplary step 710, the user may initiate a balloon inflation process, for example by pressing and holding a balloon actuator (e.g., balloon actuator 410) to the signal inflation system 170 to actuate its motor to depress syringe 174 to force fluid (e.g., saline) through the handle 400 and into the balloon 640. During step 720, the user may monitor (e.g., by viewing real-time data being charted on tablet 470 similar to that shown in FIG. 13B) pressure within the inflation line compared to the volume of inflation media pass to the balloon 640. In some examples, a critical pressure threshold PT may be set, and step 730 represents the scenario in which the critical pressure threshold PT is achieved before cracking or fracturing of the surgical prosthetic heart valve is achieved. If step 730 is reached, in step 740 the inflation of balloon 640 may be discontinued, for example either automatically by the inflation system 170, or manually by the user taking pressure off the balloon actuator 410. In step 750, the balloon 640 may be deflated and removed from the patient and a larger balloon size chosen. After the larger balloon size is chosen, the process may begin again at step 700 in another attempt at cracking the surgical prosthetic heart valve. In some examples, whether or not a critical pressure threshold PT is set, inflation of the balloon 640 may continue until cracking of the prosthetic surgical heart valve is achieved, as represented by step 760. Cracking of the prosthetic surgical heart valve may be detected, for example, by manual viewing of the pressure-volume curve and recognizing a rapid pressure drop at the end of the third stage S3 of inflation, or automatically by the inflation system 170 upon detecting a threshold level of pressure drop as volume continues to increase. After cracking of the surgical prosthetic heart valve is detected, inflation of the balloon 740 may stop at step 770, for example either automatically by the inflation system 170, or manually by the user taking pressure off the balloon actuator 410, to avoid increased risk of rupturing the patient's annulus. After inflation of the balloon 640 stops at step 770, the balloon 640 may be actively deflated, for example by pressing the balloon actuator 410 in the opposite direction, and the balloon catheter may be removed from the patient in step 780. After the balloon catheter is removed from the patient, an expandable prosthetic heart valve may be implanted into the cracked surgical prosthetic heart valve. In one example, method 200 (e.g., as shown and described in connection with FIG. 11) may be performed to achieve the valve implantation, with the exception that method 200 is performed as a valve-in-valve implantation. However, it should be understood that any other transcatheter valve replacement (including self-expandable or balloon-expandable valves, for example) may be suitable for the valve-in-valve implantation following cracking of the surgical prosthetic heart valve. Further, although the cracking procedure using the balloon 640 with one or more stress concentrator(s) 660 is described above in the context of monitoring real-time pressure-volume data to confirm cracking of the surgical prosthetic heart valve, it should be understood that the example of the balloon 640 with stress concentrators 660 may be used in a more traditional valve cracking procedure that does not rely on the type of data shown and described in connection with FIG. 13B.

In at least some examples described above, the procedure to perform the cracking or fracturing of a previously-implanted surgical heart valve is a performed as a separate procedure in the sense that the balloon catheter used for valve cracking is removed from the patient before another catheter (e.g., delivery catheter 130) is inserted into the patient as part of the follow-on transcatheter valve-in-valve implant procedure. However, in some examples, it may be desirable to have a single catheter or single catheter system that can perform both the valve cracking and the follow-on valve-in-valve implant, for example to reduce the amount of time needed to complete the procedures and to reduce the number of components needs to complete the procedures.

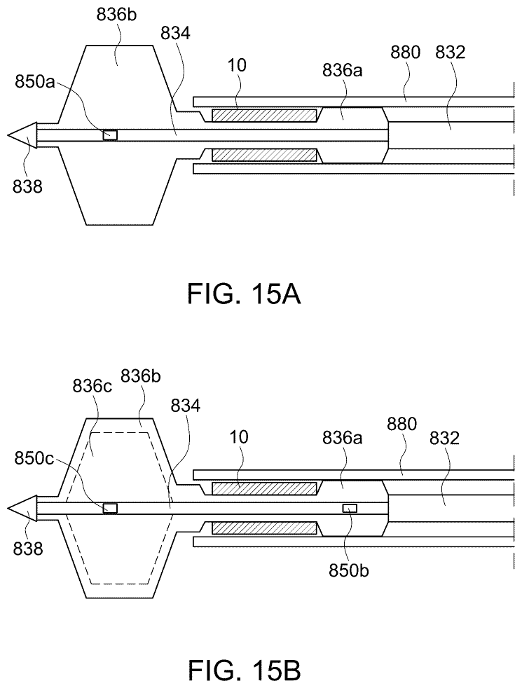

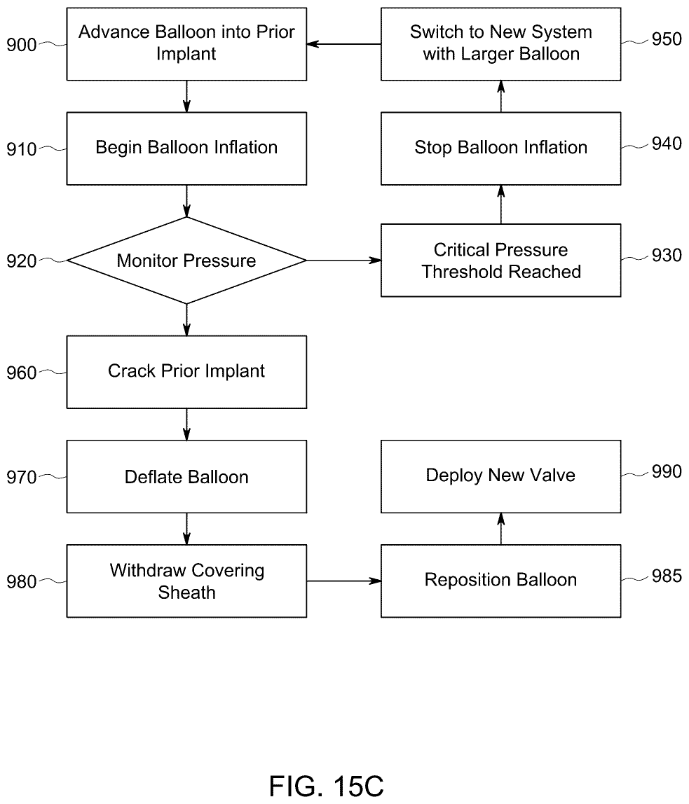

Referring now in addition to FIG. 15A, FIG. 15A is an example of a distal end of a delivery system configured to crack a prosthetic surgical valve and to deliver a new prosthetic heart valve in a valve-in-valve procedure. For example, the configuration shown and described in connection with FIG. 15A may be used as part of delivery system 100, with the exception that the distal end has a different configuration (e.g. compare FIG. 15A to the configuration of FIG. 6). In other words, the components shown and described in connection with FIG. 15A in some examples may be part of the delivery system 100 shown and described in connection with FIG. 4, with the distal end of the delivery system shown in FIG. 6 replaced with that shown in FIG. 15A.

The example of FIG. 15A includes an outer catheter 832 having a distal end that terminates at a balloon (e.g., a semi-compliant balloon or a non-compliant balloon), and the balloon may generally include a proximal portion 836a (which may be similar or identical to proximal pillowed portion 136a of balloon 136), a distal portion 836b (which may be similar or identical to distal pillowed portion 136b of balloon 136), and a central portion configured to receive a prosthetic heart valve (e.g., prosthetic heart valve 10) received thereon in a crimped condition. In some examples, a sensor, such a pressure sensor 850a, may be positioned within the fluid flow line leading to the balloon, for example similar or identical to pressures sensors 450 or 650. An inner catheter 834, which in some examples may be a guidewire catheter, may extend from the handle (e.g., handle 110), through the outer catheter 832, to a distal atraumatic tip 838. In the particular state illustrated in FIG. 15A, the distal balloon portion 836b has been expanded. However, it should be understood that when the distal balloon portion 836b is in the uninflated state, it may form a pillowed portion or shoulder similar to distal pillow portion 836b. A main difference between the embodiment of FIG. 15A compared to that of FIG. 6 is that a covering sheath may overlie portions of the distal end of the delivery device, such as the proximal balloon portion 836a and the crimped prosthetic heart valve 10, but not the distal balloon portion 836b (or not a significant amount of the distal balloon portion 836b. With this configuration, the distal end of the delivery device may be advanced through the vasculature into the previously implanted prosthetic surgical heart valve so that the distal balloon portion 836b is positioned at least partially within the surgical prosthetic heart valve. In this example, while the covering sheath 880 covers both the proximal balloon portion 836a and the prosthetic heart valve 10, the inflation media may be forced into the balloon (e.g., using inflation system 170 or any other suitable mechanism). As inflation media enters the balloon, the proximal balloon portion 836a and the center balloon portion inside of the prosthetic heart valve 10 are unable to expand because the covering sheath 880 restricts such expansion. However, the distal balloon portion 836b, which may be mostly or fully unrestricted by the covering sheath 880, is capable of expanding, for example as shown in FIG. 15A. In some examples, the balloon may be generally formed as a semi-compliant balloon, but the distal balloon portion 836b may be coated with a material such as nylon to achieve less compliance at the distal balloon portion 836b, which may be useful for valve cracking. The distal balloon portion 836b may be expanded, using conventional techniques (e.g., manual use of a syringe), using techniques similar or identical to those described in connection with FIGS. 13A-13C, and with or without stress concentrators such as those shown and described in connection with FIGS. 14A-E, in order to crack or fracture the surgical prosthetic heart valve. However, after the surgical prosthetic heart valve has been cracked, instead of removing the delivery device and inserting a new delivery device with the new prosthetic heart valve, prosthetic heart valve 10 may already be positioned adjacent to the native valve annulus. In other words, in some examples, after the surgical prosthetic heart valve has been fractured, the covering sheath 880 may be withdrawn proximally beyond the proximal balloon portion 836a (e.g., with or without first deflating the distal balloon portion 836b), and the delivery device may be positioned so that the crimped prosthetic heart valve 10 (which is now uncovered) is at a desired depth and orientation relative to the native heart valve annulus and/or the cracked surgical heart valve. Then, inflation media may again be passed to the balloon, such that the entire balloon expands to deploy the prosthetic heart valve 10 within the previously-implanted surgical prosthetic heart valve. Thus, with the particular configuration of FIG. 15A, the previously implanted surgical prosthetic heart valve may be fractured using the same balloon (or a portion thereof) that is used immediately thereafter to deploy the prosthetic heart valve 10 in the fractured surgical heart valve, allowing for significant efficiency performing both procedures.

In the example of FIG. 15A, as the balloon is pressurized during the cracking procedure, even though only the distal balloon portion 836b inflates, the entire balloon may become pressurized which may apply force against the prosthetic heart valve 10, even though the covering sheath 880 prevents the prosthetic heart valve 10 from expanding. Exposing the prosthetic heart valve 10 to this balloon pressurization while covering sheath 880 restricts its expansion may not always be desirable. In the example of FIG. 15B, this potential problem may be avoided by providing a second, separate balloon 836c internal to the distal balloon portion 836b. With this configuration, during the cracking procedure, inflation media may be delivered through a separate inflation lumen (e.g., running through a wall of the inner catheter 834) that leads into the interior of the second balloon 836c. With this configuration, the second balloon 836c can be inflated, which will result in the distal balloon portion 836b also expanding, but without pressuring the portions of the balloon maintained within the covering sheath 880 during the cracking procedure. In other words, the use of the second balloon 836c in some examples may isolate the balloon pressure for cracking to areas distal of the prosthetic heart valve 10. If two balloons are used, in some examples, the second balloon 836c may be a non-compliant balloon while the primary balloon (which may include proximal balloon portion 836a, distal balloon portion 836b, and the central balloon portion over which the prosthetic heart valve 10 is crimped) may be a semi-compliant balloon. A single fluid reservoir in some examples may be fluidly coupled to both balloons (e.g., via valving mechanisms to selectively delivery fluid to the balloons), but in other examples individual fluid reservoirs may each be coupled to the respective balloons. After the cracking is completed, the prosthetic heart valve 10 may be implanted in substantially the same way as described above in connection with FIG. 15A. In some examples, if the second balloon 836c is included and is part of a fluid flow path that is isolated form the main fluid flow path to the primary balloon, a first pressure sensor 850b may be provided within the main inflation lumen fluid path (e.g., within proximal balloon portion 836a), and a second pressure sensor 850c may be provided within the inflation lumen fluid path for the secondary balloon 836c. With this configuration, pressure readings may be taken from the second sensor 850c during the cracking procedure, while pressure readings may be taken from the first sensor 850b during the valve-in-valve implantation procedure.

Now referring in addition to FIG. 15C, FIG. 15C is a flowchart providing an example method of use relating to the configuration(s) of FIGS. 15A-15B. As shown in FIG. 14C, in a first exemplary step 900, a user may advance the distal end of the balloon catheter (which may include a handle similar or identical to handle 110 attached to an inflation system similar or identical to inflation system 170) into a patient while the new prosthetic heart valve 10 is maintained in a crimped condition between the deflated proximal balloon portion 836a and distal balloon portion 836b. The distal end of the balloon catheter may be advanced until the distal balloon portion 836b is positioned at least partially inside a previously-implanted prosthetic surgical heart valve (e.g., valve 300 of FIG. 10A) while the distal balloon portion 836b is in a deflated condition. Once the deflated distal balloon portion 836b is within the prosthetic surgical heart valve, in exemplary step 910, the user may initiate a balloon inflation process, for example by pressing and holding a balloon actuator (e.g., balloon actuator 120) to the signal inflation system 170 to actuate its motor to depress syringe 174 to force fluid (e.g., saline) through the handle 110. This step may be performed while the distal balloon portion 836b is uncovered by the covering sheath 880, but while the new prosthetic heart valve 10 and the proximal balloon portion 836a are covered by the covering sheath 880. If the device includes only a single balloon, as in FIG. 15A, this inflation step passes fluid directly into the distal balloon portion 836b. If the device includes a secondary balloon 836c within the distal balloon portion 836b, this inflation step passes fluid directly into the secondary balloon 836c. It should be understood that if distal balloon portion 836b includes stress concentrator features (e.g., similar to those shown and described in connection with FIGS. 14A-14D), an imaging and repositioning step similar to step 705 of FIG. 14E may be performed prior to the inflation.

In some examples, during step 920, the user may monitor (e.g., by viewing real-time data being charted on tablet 470 similar to that shown in FIG. 13B) pressure within the inflation line compared to the volume of inflation media pass to the balloon. For example, pressure readings may be taken from pressure sensor 850a if a single balloon similar to that shown in FIG. 15A is used, or from pressure sensor 850c is a secondary balloon similar to that shown in FIG. 15B is included. In some examples, a critical pressure threshold PT may be set, and step 930 represents the scenario in which the critical pressure threshold PT is achieved before cracking or fracturing of the surgical prosthetic heart valve is achieved. If step 930 is reached, in step 940 the inflation may be discontinued, for example either automatically by the inflation system 170, or manually by the user taking pressure off the balloon actuator 120. In step 950, the balloon may be deflated and removed from the patient and a larger balloon size chosen. After the larger balloon size is chosen, the process may begin again at step 900 in another attempt at the procedure.

In some examples, whether or not a critical pressure threshold PT is set, inflation of the balloon distal balloon portion 836b (and/or secondary balloon 836c) may continue until cracking of the prosthetic surgical heart valve is achieved, as represented by step 960. Cracking of the prosthetic surgical heart valve may be detected, for example, by manual viewing of the pressure-volume curve and recognizing a rapid pressure drop at the end of the third stage S3 of inflation, or automatically by the inflation system 170 upon detecting a threshold level of pressure drop as volume continues to increase. After cracking of the surgical prosthetic heart valve is detected, inflation of the distal balloon portion 836b (and/or secondary balloon 836c) may stop at step 970, for example either automatically by the inflation system 170, or manually by the user taking pressure off the balloon actuator 120, to avoid increased risk of rupturing the patient's annulus. In step 980, the covering sheath 880 may be withdrawn (either with or without first actively deflating the distal balloon portion 836b (and/or secondary balloon 836c), so that the covering sheath 880 does not cover the new prosthetic heart valve 10 or the proximal balloon portion 836a. At step 985 (which may occur before or after the covering sheath 880 is withdrawn), the distal end of the balloon catheter may be repositioned so that the new prosthetic heart valve 10 is in a desired alignment with respect to the native heart valve and/or the cracked prosthetic surgical heart valve. Then, at step 990, the new prosthetic heart valve 10 may be deployed by inflating the balloon. It should be understood that the positioning of step 985 and deployment of step 990 may be similar or identical to steps 210 through 216 shown and described in connection with method 200. In a final step, similar to step 218, the balloon may be deflated and the balloon catheter may be removed from the patient.

In the example described above, at step 985, the catheter is described as being repositioned (e.g., advanced further into the left ventricle) in order to achieve desired alignment of the new prosthetic heart valve 10 within the fractured, previously-implanted prosthetic valve. However, in some examples, it may be desirable to avoid the necessity to conduct this repositioning step 985. Thus, in some examples, the balloon catheter shown in FIG. 15A or FIG. 15B may include a feature that advances the crimped prosthetic heart valve 10 distally onto the same balloon that was already inflated (e.g., the distal balloon portion 836b), so the same balloon portion could be re-inflated, without repositioning, to deploy the prosthetic heart valve 10. For example, a pusher may be provided to push the prosthetic heart valve 10 forward after deflating the distal balloon portion 836b following cracking of the surgical valve. In other examples, a portion of the distal balloon portion 836b may be withdrawn proximally into the delivery system to shorten the effective length of the delivery system, which would allow the delivery system to be advanced into the left ventricle during the repositioning step 985, without the distalmost end of the delivery device advancing too far into the left ventricle.

Some of the techniques described herein, including computer-related and processor-related techniques relating to operation of the inflation system 170, the computer 460, and/or the tablet 470, may be implemented in some examples at least in part by one or more special-purpose computing devices. The disclosure described below may apply to each of the inflation system 170, the computer 450, and/or the tablet 470 as either individual components or components working in unison. The special-purpose computing devices may be hard-wired to perform one or more techniques described herein, including combinations thereof. Alternatively and/or in addition, the one or more special-purpose computing devices may include digital electronic devices such as one or more application-specific integrated circuits (ASICs) or field-programmable gate arrays (FPGAs) that are persistently programmed to perform the techniques. Alternatively and/or in addition, the one or more special-purpose computing devices may include one or more general-purpose hardware processors programmed to perform the techniques described herein pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices, and/or any other device that incorporates hard-wired or program logic to implement the techniques.

FIG. 16 is a block diagram that illustrates a computer system upon which an example may be implemented. The computer system 1000 may include a bus 1002 or other communication mechanism for communicating information, and one or more hardware processors 1004 coupled with bus 1002 for processing information, such as computer instructions and data. The processor/s 1004 may include one or more general-purpose microprocessors, graphical processing units (GPUs), coprocessors, central processing units (CPUs), and/or other hardware processing units.

The computer system 1000 may also include one or more units of main memory 1006 coupled to the bus 1002, such as random-access memory (RAM) or other dynamic storage, for storing information and instructions to be executed by the processor/s 1004. Main memory 1006 may also be used for storing temporary variables or other intermediate information during execution of instructions to be executed by the processor/s 1004. Such instructions, when stored in non-transitory storage media accessible to the processor/s 1004, may turn the computer system 1000 into a special-purpose machine that is customized to perform the operations specified in the instructions. In some embodiments, main memory 1006 may include dynamic random-access memory (DRAM) (including but not limited to double data rate synchronous dynamic random-access memory (DDR SDRAM), thyristor random-access memory (T-RAM), zero-capacitor (Z-RAM™)) and/or non-volatile random-access memory (NVRAM).

The computer system 1000 may further include one or more units of read-only memory (ROM) 1008 or other static storage coupled to the bus 1002 for storing information and instructions for the processor/s 1004 that are either always static or static in normal operation but reprogrammable. For example, the ROM 1008 may store firmware for the computer system 1000. The ROM 1008 may include mask ROM (MROM) or other hard-wired ROM storing purely static information, programmable read-only memory (PROM), erasable programmable read-only memory (EPROM), electrically-erasable programmable read-only memory (EEPROM), another hardware memory chip or cartridge, or any other read-only memory unit.

One or more storage devices 1010, such as a magnetic disk or optical disk, is provided and coupled to the bus 1002 for storing information and/or instructions. The storage device/s 1010 may include non-volatile storage media such as, for example, read-only memory, optical disks (such as but not limited to compact discs (CDs), digital video discs (DVDs), Blu-ray discs (BDs)), magnetic disks, other magnetic media such as floppy disks and magnetic tape, solid-state drives, flash memory, optical disks, one or more forms of non-volatile random-access memory (NVRAM), and/or other non-volatile storage media. The computer system 1000 may be coupled via the bus 1002 to one or more input/output (I/O) devices 1012. For example, the I/O device/s 1012 may include one or more displays for displaying information to a computer user, such as a cathode ray tube (CRT) display, a Liquid Crystal Display (LCD) display, a Light-Emitting Diode (LED) display, a projector, and/or any other type of display.

The I/O device/s 1012 may also include one or more input devices, such as an alphanumeric keyboard and/or any other keypad device. In some examples, the balloon actuators described herein may be an input device. The one or more input devices may also include one or more cursor control devices, such as a mouse, a trackball, a touch input device, or cursor direction keys for communicating direction information and command selections to the processor 1004 and for controlling cursor movement on another I/O device (e.g. a display). A cursor control device typically has degrees of freedom in two or more axes, (e.g. a first axis x, a second axis y, and optionally one or more additional axes z), that allows the device to specify positions in a plane. In some embodiments, the one or more I/O device/s 1012 may include a device with combined I/O functionality, such as a touch-enabled display.

Other I/O device/s 1012 may include a fingerprint reader, a scanner, an infrared (IR) device, an imaging device such as a camera or video recording device, a microphone, a speaker, an ambient light sensor, a pressure sensor, an accelerometer, a gyroscope, a magnetometer, another motion sensor, or any other device that can communicate signals, commands, and/or other information with the processor/s 1004 over the bus 1002.

The computer system 1000 may implement the techniques described herein using customized hard-wired logic, one or more ASICs or FPGAs, firmware, and/or program logic which, in combination with the computer system causes or programs, causes computer system 1000 to be a special-purpose machine. In some examples, the techniques herein are performed by the computer system 1000 in response to the processor/s 1004 executing one or more sequences of one or more instructions contained in main memory 1006. Such instructions may be read into main memory 1006 from another storage medium, such as the one or more storage device/s 1010. Execution of the sequences of instructions contained in main memory 1006 causes the processor/s 1004 to perform the process steps described herein. In alternative embodiments, hard-wired circuitry may be used in place of or in combination with software instructions.

The computer system 1000 may also include one or more communication interfaces 1018 coupled to the bus 1002. The communication interface/s 1018 provide two-way data communication over one or more physical or wireless network links 1020 that are connected to a local network 1022 and/or a wide area network (WAN), such as the Internet. For example, the communication interface/s 1018 may include an integrated services digital network (ISDN) card, cable modem, satellite modem, or a modem to provide a data communication connection to a corresponding type of telephone line. Alternatively and/or in addition, the communication interface/s 1018 may include one or more of: a local area network (LAN) device that provides a data communication connection to a compatible local network 1022; a wireless local area network (WLAN) device that sends and receives wireless signals (such as electrical signals, electromagnetic signals, optical signals or other wireless signals representing various types of information) to a compatible LAN; a wireless wide area network (WWAN) device that sends and receives such signals over a cellular network; and other networking devices that establish a communication channel between the computer system 1000 and one or more LANs 1022 and/or WANs. The network link/s 1020 typically provides data communication through one or more networks to other data devices. For example, the network link/s 1020 may provide a connection through one or more local area networks 1022 (LANs) to one or more host computers 1024 or to data equipment operated by an Internet Service Provider (ISP) 1026. The ISP 1026 provides connectivity to one or more wide area networks 1028, such as the Internet. The LAN/s 1022 and WAN/s 1028 use electrical, electromagnetic, or optical signals that carry digital data streams. The signals through the various networks and the signals on the network link/s 1020 and through the communication interface/s 1018 are example forms of transmission media, or transitory media.

The term “storage media” as used herein refers to any non-transitory media that stores data and/or instructions that cause a machine to operate in a specific fashion. Such storage media may include volatile and/or non-volatile media. Storage media is distinct from but may be used in conjunction with transmission media. Transmission media participates in transferring information between storage media. For example, transmission media includes coaxial cables, copper wire and fiber optics, including traces and/or other physical electrically conductive components that comprise the bus 1002. Transmission media can also take the form of acoustic or light waves, such as those generated during radio-wave and infra-red data communications.

Various forms of media may be involved in carrying one or more sequences of one or more instructions to the processor 1004 for execution. For example, the instructions may initially be carried on a magnetic disk or solid-state drive of a remote computer. The remote computer can load the instructions into its main memory 1006 and send the instructions over a telecommunications line using a modem. A modem local to the computer system 1000 can receive the data on the telephone line and use an infra-red transmitter to convert the data to an infra-red signal. An infra-red detector can receive the data carried in the infra-red signal and appropriate circuitry can place the data on the bus 1002. The bus 1002 carries the data to main memory 1006, from which the processor 1004 retrieves and executes the instructions. The instructions received by main memory 1006 may optionally be stored on the storage device 1010 either before or after execution by the processor 1004.

The computer system 1000 can send messages and receive data, including program code, through the network(s), the network link 1020, and the communication interface/s 1018. In the Internet example, one or more servers 1030 may transmit signals corresponding to data or instructions requested for an application program executed by the computer system 1000 through the Internet 1028, ISP 1026, local network 1022 and a communication interface 1018. The received signals may include instructions and/or information for execution and/or processing by the processor/s 1004. The processor/s 1004 may execute and/or process the instructions and/or information upon receiving the signals by accessing main memory 1006, or at a later time by storing them and then accessing them from the storage device/s 1010.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of fracturing a previously-implanted prosthetic surgical heart valve, the method comprising:
advancing a non-compliant balloon of a balloon catheter through a patient's vasculature until the balloon is positioned at least partially within the previously-implanted prosthetic surgical heart valve;
advancing inflation media through an inflation lumen and into the balloon to inflate the balloon;
measuring a pressure within the inflation lumen during advancing the inflation media;
measuring a volume of inflation media advanced through the inflation lumen and into the balloon; and
monitoring a relationship between the measured pressure and the measured volume of inflation, as the inflation media is advanced into the balloon, to identify distinct phases of balloon inflation.

2. The method of claim 1, wherein monitoring the relationship between the measured pressure and the measured volume of inflation includes viewing a real-time pressure-volume curve provided on a display based on the measured pressure and the measured volume.

3. The method of claim 2, wherein identifying distinct phases of balloon inflation includes identifying a first phase of inflation corresponding to initial balloon filling phase in which the measured pressure stays substantially constant as the measured volume increases, the pressure-volume curve having a first slope during the first phase of inflation.

4. The method of claim 3, wherein identifying distinct phases of balloon inflation includes identifying a second phase of inflation corresponding to additional balloon filling phase in which the measured pressure increases as the measured volume increases, the pressure-volume curve having a second slope during the second phase of inflation, the second slope being greater than the first slope.

5. The method of claim 4, wherein identifying distinct phases of balloon inflation includes identifying a third phase of inflation corresponding to contact between the balloon and the previously-implanted prosthetic surgical valve in which the measured pressure increases as the measured volume increases, the pressure-volume curve having a third slope during the third phase of inflation, the third slope being greater than the second slope.

6. The method of claim 5, wherein identifying distinct phases of balloon inflation includes identifying a fourth phase of inflation corresponding to a frame of the previously-implanted prosthetic surgical valve being fractured in which the measured pressure decreases as the measured volume increases.

7. The method of claim 6, further comprising stopping advancing inflation media through the inflation lumen after identifying the fourth phase of inflation.

8. The method of claim 7, wherein advancing inflation media through the inflation lumen and into the balloon to inflate the balloon is performed with a motorized inflation system, and the motorized inflation system automatically stops advancing inflation media through the inflation lumen after identifying the fourth phase of inflation.

9. The method of claim 2, further comprising identifying a critical balloon pressure threshold.

10. The method of claim 9, further comprising stopping advancing inflation media through the inflation lumen if the measured pressure reaches the critical pressure balloon pressure threshold before the balloon fractures the previously-implanted prosthetic surgical heart valve.

11. A balloon catheter system configured to fracture a previously-implanted prosthetic heart valve, the balloon catheter system comprising:
a motorized inflation system configured to (i) operably couple to a syringe filled with inflation media, (ii) pressurize the syringe to advance inflation media out of the syringe, and (iii) measure a volume of inflation media advanced out of the syringe;
a balloon catheter including a balloon at a distal end thereof, the balloon configured to be in fluid communication with the syringe via an inflation lumen when the syringe is operably coupled to the motorized inflation system and when the balloon catheter is operably coupled to the syringe;
a pressure sensor positioned within a pathway of the inflation lumen configured to measure a pressure within the inflation lumen;
a computer system operably coupled to the motorized inflation system; and
a display operably coupled to the computer system, the display configured to display a real-time pressure-volume curve based on the measured pressure and the measured volume.

12. The balloon catheter system of claim 11, wherein the computer system is configured to identify at least four distinct phases of balloon inflation.

13. The balloon catheter system of claim 12, wherein a first phase of the at least four distinct phases of balloon inflation corresponds to initial balloon filling phase in which the measured pressure stays substantially constant as the measured volume increases, the pressure-volume curve having a first slope during the first phase of inflation.

14. The balloon catheter system of claim 13, wherein a second phase of the at least four distinct phases of balloon inflation corresponds to an additional balloon filling phase in which the measured pressure increases as the measured volume increases, the pressure-volume curve having a second slope during the second phase of inflation, the second slope being greater than the first slope.

15. The balloon catheter system of claim 14, wherein a third phase of the at least four distinct phases of balloon inflation corresponds to contact between the balloon and the previously-implanted prosthetic surgical valve in which the measured pressure increases as the measured volume increases, the pressure-volume curve having a third slope during the third phase of inflation, the third slope being greater than the second slope.

16. The balloon catheter system of claim 15, wherein a fourth phase of the at least four distinct phases of balloon inflation corresponds to a frame of the previously-implanted prosthetic surgical valve being fractured in which the measured pressure decreases as the measured volume increases.

17. The balloon catheter system of claim 16, wherein the computer system is configured to instruct the motorized inflation system to stop advancing inflation media out of the syringe upon detecting the fourth phase of balloon inflation.

18. The balloon catheter system of claim 11, wherein the computer system is configured to receive a critical balloon pressure threshold set point.

19. The balloon catheter system of claim 18, wherein the computer system is configured to instruct the motorized inflation system to stop advancing inflation media out of the syringe upon detecting the measured pressure has reached the critical balloon pressure threshold set point.

20. A balloon catheter system configured to fracture a previously-implanted prosthetic heart valve, the balloon catheter system comprising:
a balloon catheter including a balloon at a distal end thereof, the balloon configured to be in fluid communication with a fluid reservoir via an inflation lumen; and
at least one stress concentrator coupled to an outer surface of the balloon, the at least one stress concentrator extending in a proximal-to-distal direction of the balloon, wherein the balloon has a length in the proximal-to-distal direction, and the at least one stress concentrator extends at least 50% of the axial length of the balloon, wherein upon inflation of the balloon within the previously-implanted prosthetic heart valve, the at least one stress concentrator is configured to contact the previously-implanted prosthetic heart valve prior to the outer surface of the balloon contacting the previously-implanted prosthetic heart valve.
